(19) United States
(12) Patent Application Publication
(10) Pub. No.: US 2022/0051592 A1
(43) Pub. Date: Feb. 17, 2022

(54) RESPIRATORY SYSTEM SIMULATOR SYSTEMS AND METHODS

(71) Applicant: Massachusetts Institute of Technology, Cambridge, MA (US)

(72) Inventors: Lydia Bourouiba, Cambridge, MA (US); Yongji Wang, Roxbury, MA (US); Rishabh Datta, Cambridge, MA (US)

(21) Appl. No.: 17/405,007

(22) Filed: Aug. 17, 2021

Related U.S. Application Data
(60) Provisional application No. 63/066,786, filed on Aug. 17, 2020.

(51) Int. Cl. G09B 23/30; G09B 23/32

(52) U.S. Cl. CPC G09B 23/303; G09B 23/32

(57) ABSTRACT

Respiratory System Similation Systems that mimic human or other animal exhalation events are disclosed. Exhalation events that can be reproduced include coughing, sneezing, breathing, talking, gagging, panting, and singing. Air flow and airway systems cooperate to eject a gas cloud comprising the at least some air produced from the air flow system and one or more of a plurality of droplets, solid residues, or aerosols. The exhalation emission systems can be used in testing emissions in various environments, medical, and protective equipment usage situations. Methods related to the same are also disclosed.

### CROSS REFERENCE TO RELATED APPLICATIONS

This application claims priority to and the benefit of U.S. Provisional Patent Application No. 63/066,786, entitled “Exhalation Emission Simulator Systems and Methods,” filed Aug. 17, 2020, the content of which is incorporated by reference herein in its entirety.

### FIELD

The present disclosure relates to systems and methods for simulating exhalation emissions, and more particularly relates to systems and methods that account for both liquid and gas phases of emissions when performing actions such as coughing, sneezing, breathing, talking, and/or singing, as well as interventions, devices and physiological and disease processes that induce produce, modify or interfere with such emissions.

### BACKGROUND

Airborne infectious diseases are a serious burden on society and a serious threat to the livestock industry and animal species around the globe. In airborne infectious diseases, the transmission of pathogens is mainly induced by respiratory events, such as exhalations, talking/vocalizations, singing, gagging, coughing or sneezing. However, quantitative analyses of pathogen transmission via various respiratory events remain limited.

The difficulty lies in the limitation of exhalation samples that can be reproducibly gathered from human subjects or animals. Current emission systems and methods simply represent exhalations through overly simplistic and unrealistic spray atomization or nebulization processes. Additionally, they do not represent the geometric configuration or mechanical properties of the respiratory tract, including the trachea, oropharynx, mouth and nose that contribute to shaping the multiphase flow characteristics, including the opening angle of the (point-source) of the turbulent multiphase gas cloud emissions. Furthermore, the duration and strength (overpressure, volume and liquid volume fraction exhaled) of the respiratory air flow, and the fluid properties of the mucosalivary fluid all significantly affect the regimes of fragmentation, leading to the generation of respiratory droplets over a wide range of sizes and speeds. Until now, no consideration has been given to these effects in the design of an apparatus to reliably and reproducibly generate realistic simulated respiratory events.

Due to these limitations, it has been difficult to ascertain how far pathogens can be transmitted, how long they can survive in and how far they can be transported through the air, to what extent the transmission characteristics change under different environmental conditions, how far people or livestock should be separated to substantially reduce the risk of transmission, what types of aerosol-generating medical procedures increase the risk of pathogen emission and disease transmission, and which kinds of infection control devices, such as different types of masks, face shields, or air flow diversion tactics, are most effective in reducing the risk of disease transmission.

Accordingly, there is a need for systems and methods that consistently and physiologically accurately reproduce the multiphase turbulent air flow and multiscale spray properties for a range of regimes of controlled exhalations such normal tidal breathing, panting, talking/vocalizing at different intensities, singing, gagging, coughing or sneezing across the spectrum of health and disease states.

### SUMMARY

The present disclosure is directed to systems and methods that allow for an accurate portrayal and realization of the multiphase turbulent gas cloud nature of human and animal exhalation emission events, such as coughing, sneezing, breathing, vocalizations/talking, gagging, and/or singing. These systems and methods allow for testing of realistic exhalation emission events to determine distribution of exhaled air, fluid droplets, and aerosols travel when injected into an environment, as well as the efficacy of various protective equipment aimed at preventing or reducing the transmission of pathogens from such exhalation.

Disclosed systems, referred to here as a Respiratory System Simulator System(s) (RSSS), and methods provide repeatable and controllable reproduction of exhalation emission events by incorporating both airflow systems and airway systems that allow the profile of the generated air flow, as well as the incorporation of a generation of fluid droplets in the airflow to be prescribed by controlling and adjusting a number of parameters in the RSSS to achieve a desired exhalation emission event. The control and adjustment provided in the RSSS allow for a more effective analyses of the spatiotemporal distributions of such exhalation emission events and allow for more effective and repeatable testing of various risk-mitigation strategies, sometimes referred to as source protection devices, or personal protective equipment such as masks, face shields, or protective materials an barriers, intended to limit the range of such emissions.

More particularly, by employing an RSSS with an airflow system that can control one or more of flow pattern, momentum, heat, and humidity of an airflow and an airway system with conduits designed to replicate respiratory airways of an organism, a multiphase flow is generated that represents, or mimics, a desired exhalation event from the RSSS.

One embodiment of a system for exhalation emission includes an air flow system and an airway system. The air flow system is configured to have a gas flow exit from it. The gas flow has a prescribed flow pattern and a prescribed momentum that corresponds to one or more exhalation events. The airway system has an airway in fluid communication with the air flow system to receive the gas flow from the air flow system and at least one exit orifice. The airway system is configured to mimic a respiratory tract. The system for exhalation emission is configured such that at least some portion of the gas flow produced from the air flow system passes through the airway and out of the at least one exit orifice in a multiphase gas cloud. The multiphase gas cloud includes at least some portion of the gas flow produced from the air flow system and one or more of a plurality of droplets, solid residues, or aerosols.

The one or more of the plurality of droplets, solid residues, or aerosols can be produced by the airway system due to: (1) the gas flow interacting with a liquid coating disposed in the airway in conjunction with mimicking a respiratory tract; and/or (2) the gas flow interacting with liquid injected into the airway system.

The air flow system can include a gas containing chamber. The gas containing chamber can include a temperature stabilizing unit and/or a humidifier/dehumidifier system. In embodiments that include the temperature stabilizing unit, the unit can be coupled to an exterior of the gas containing chamber and can be configured to place the gas flow in a prescribed temperature range. In embodiments that include the humidifier/dehumidifier system, the system can be configured to place the gas flow in a prescribed humidity range. The air flow system can further include at least one pressure or flow regulator that can be configured to deliver input air into the gas containing chamber.

The air flow system can include an exhalation event control system that can be disposed between at least one of the air flow system and the airway system. The exhalation event control system can be configured to generate a pulse of air from air received from a gas containing chamber and can deliver the pulse of air to the airway system at the prescribed flow pattern. The prescribed flow pattern can include at least one of a velocity of air flow, a duration of air flow, or a magnitude modulation of air flow commensurate with a desired exhalation event from the one or more exhalation events. The exhalation event control system can also be configured to deliver the pulse of air to the airway system at a prescribed volume and/or can have at least one pulse generator or valve. The air flow system can be configured to achieve a prescribed buoyancy of the multiphase gas cloud a certain distance from the airway system.

The airway system can include a fluid disposed in the airway. The fluid can have rheological properties akin to a mucosalivary fluid. The fluid can line at least a portion of the airway. The fluid can be configured to be sheared by the gas flow produced from the air flow system that passes through the airway such that the fluid becomes the one or more of the plurality of droplets, the solid residues, or the aerosols in the multiphase gas cloud.

The system can include a fluid inlet and a fluid outlet, each in fluid communication with the airway. The system can be configured to pass the fluid configured to mimic mucosalivary fluid from the inlet, into the airway, and out of the outlet. In some embodiments the at least one exit orifice can include at least two exit orifices. For example, a first exit orifice can be configured to mimic a shape and/or mechanical properties of a human nose and a second exit orifice can be configured to mimic a shape and/or mechanical properties of a mouth. In some embodiments the exit orifice(s) can include a mouthpiece configured to mimic at least one of shape or mechanical properties of a human mouth. More generally, the at least one exit orifice can be configured to replicate a shape and/or mechanical properties of at least one of a nose or a mouth. Liquids of a range of rheological properties can be configured to be loaded into the airway system through at least one liquid inlet. The rheological properties of the liquid can be in a range of rheological properties of bodily liquids in health or disease.

The air flow and the at least one exit orifice can be configured in such a way to create a range of point-source multiphase gas clouds consistent with a range of natural respiratory emissions. In some embodiments, the air flow system can be configured to achieve a range of size distributions for at least one of the plurality of droplets, the solid residue, or the aerosol upon exit of the airway system.

One or more human exhalation events related to the system can include at least one of coughing, sneezing, breathing, talking, panting, gagging, or singing, and in at least some instances it can include coughing and/or sneezing. The exhalation event(s) can be configured to be based on at least one of different ages, different sizes, different shapes, or different medical conditions.

One embodiment of a method for generating an exhalation emission includes generating a flow of air at a prescribed flow pattern, a prescribed momentum, and one or both of a prescribed liquid or solid volume fraction, for one or more exhalation events. The method further includes directing the flow of air through an airway configured to mimic a respiratory tract such that at least some air of the flow of air exits the airway in a multiphase gas cloud. The gas cloud includes the at least some air and one or more of a plurality of droplets, solid residues, or aerosols.

Directing the flow of air through an airway configured to mimic a respiratory tract can also include causing at least one of: (1) a fluid disposed in the airway to become entrained with the at least some air of the flow of air such that the fluid becomes the one or more of the plurality of droplets, solid residues, or aerosols; or (2) injecting a fluid into the respiratory tract such that the injected fluid interacts with the flow of air to become the one or more of the plurality of droplets, solid residues, or aerosols. The fluid disposed in the airway can line at least a portion of the airway. In some such embodiments, directing the flow of air through an airway configured to mimic a respiratory tract can include shearing the fluid away from the airway to cause the fluid to become entrained with the at least some air of the flow of air. The method can include passing the fluid through the airway.

In some embodiments, the method can further include adjusting at least one of a pressure of the flow of air, the temperature of the flow of air, or the humidity of the flow of air to fall within a respective prescribed pressure range, a prescribed temperature range, or a prescribed humidity ranged based on data related to at least one of an age of the human or animal, a size of the human or animal, a shape of a human or animal, or one or more medical conditions of a human or animal. The method can also include adjusting at least one of peak flow velocity of the flow of air, a duration of the flow of air, or a volume of the flow of air based on data related to at least one of an age of the human or animal, a size of the human or animal, a shape of a human or animal, or one or more medical conditions of a human or animal.

At least some air of the flow of air that exits the airway in a gas cloud can exit through at least two exit orifices. The at least two exit orifices can include a first exit orifice that can be configured to replicate a shape and/or mechanical properties of a nose and a second orifice that can be configured to replicate a shape and/or mechanical properties of a mouth. The prescribed pressure range, the prescribed temperature range, and the prescribed humidity range of the generated flow of air can be prescribed based on one or more intended exhalation events. The one or more intended exhalation events can include at least one of coughing, sneezing, breathing, talking, gagging, panting, or singing.

### BRIEF DESCRIPTION OF THE DRAWINGS

This disclosure will be more fully understood from the following detailed description taken in conjunction with the accompanying drawings, in which:

FIG. 1 is a schematic illustration of a respiratory system simulator system of the present disclosure;

FIG. 2 is a schematic illustration of another respiratory system simulator system embodiment of the present disclosure;

FIG. 3A is a schematic illustration of an air flow system according to the disclosed embodiments, which in at least some contexts can be considered a respiratory system simulator system, the system being in a testing configuration for personal protective equipment;

FIG. 3B is a schematic illustration of the air flow system of FIG. 3A, but the system being in a testing configuration for an airway medical device;

FIG. 4 is a schematic view of an exemplary test environment for a respiratory system simulator system and personal protective equipment configuration;

FIG. 5 is a block diagram of one exemplary embodiment of a computer system for use in conjunction with the present disclosures; and

FIGS. 6A and 6B illustrate graphs of the relationship between emission droplet sizes at distances from emission orifices of humans in FIG. 6A, and the relationship between concentration and diameter of droplets for particular emission events in FIG. 6B.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. In the present disclosure, like-numbered components of various embodiments generally have similar features and functionality when those components are of a similar nature and/or serve a similar purpose. Terms like “may” and “can” are used interchangeably herein and are used in a non-exclusive manner, meaning what is described in conjunction with the same is a non-limiting embodiment with other possible elements, configurations, etc. existing in view of the present disclosures. Further, the present disclosure includes some illustrations and descriptions that include prototypes, schematic illustrations, and/or bench models. A person skilled in the art will recognize how to rely upon the present disclosure to integrate or otherwise turn the systems, devices, techniques, and methods provided for herein into a commercial product, testing apparatus, etc.

Exhalation emissions systems and related methods are disclosed herein to consistently and accurately recreate emission situations for humans or other animals across a wide range of conditions. The RSSS may comprise an air flow system and an airway system. The air flow system may be designed to produces desired air flow characteristics and is in communication with the airway system. The airway system may be designed to replicate an airway anatomy of one or more emission paths for an air flow produced by the air flow system to traverse prior to being injected into the environment as an exhalation. The RSSS system produces flow profiles by prescribing one or more of velocity of air flow, total air volume, a duration of air flow, a volumetric flow rate, or a magnitude modulation of air flow commensurate with a desired exhalation event from the one or more exhalation events. The RSSS system further achieves a range of droplet and/or solid residue/aerosol size distributions upon exit of the airway testing system. In this manner, emissions from the RSSS that replicate sneezing, coughing, exhalation, and other respiratory events may be produced.

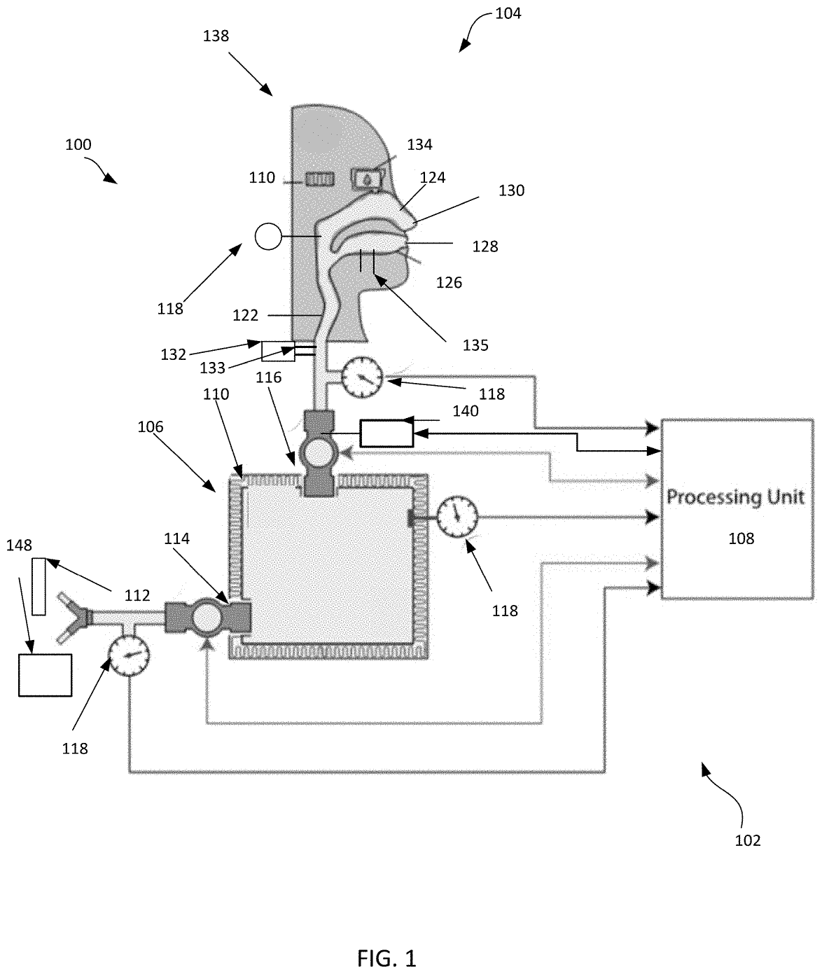

FIG. 1 illustrates an exemplary embodiment of an exhalation emission system 100.

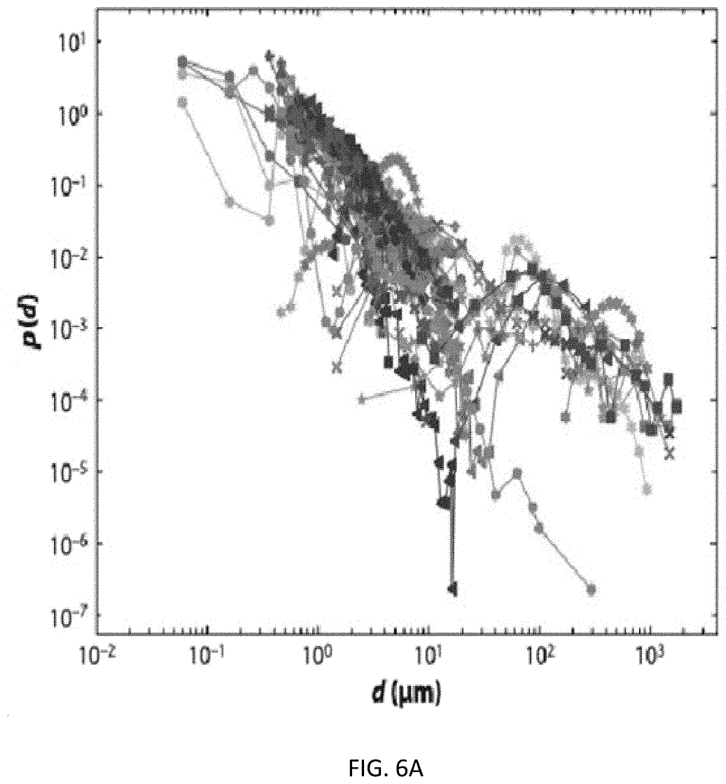

RSSS 100 includes air flow system 102 and airway system 104. Air flow system 102 may include at least a gas containing element 106, such as a pressure control chamber, and a processing unit 108. At least one temperature stabilizing unit 110, and humidification/de-humidification system including one or more humidifiers/dehumidifiers 112, may be coupled to and/or located outside of the gas containing element 106 to adjustably control temperature and humidity of an air flow. The at least one temperature stabilizing unit 110 may include at least one of heating coils or air, water, or gel temperature stabilizing jackets, and/or countercurrent heating units. A person skilled in the art will appreciate other devices or components that can be used to control temperature without departing from the spirit of the present disclosure. Gas containing element 106 may include at least one inlet 114 and outlet 116. Inlet(s) 114 can be configured for replenishing gas in the gas containing element 106, and may include an inlet valve and pressure regulator and/or resistive element, such as an actuated valve or assembly of valves as well as a gas source 148. Outlet(s) 116 can be configured for air release from the gas containing element 106 to the airway system 104 and may include an outlet valve and/or resistive element, such as an actuated valve or assembly of valves, among other features known to a person skilled in the art for controlling fluid flow, such elements more generally being considered fluid control elements or valves. One or more of sensors 118 may also be positioned at various locations of the air flow system 102, such as within or in communication with the gas containing element 106, the inlet 114, the outlet 116, and/or communication paths (e.g., conduits, tubing, pipes, etc.) disposed therebetween for sensing any of air flow, air flow velocity, pressure, temperature, humidity, and/or gas composition of an air flow, among other possible parameters that can be monitored. The processing unit 108 may be in communication with the one or more sensors 118 to adjustably control air flow, air flow velocity, pressure, temperature, humidity, and/or gas composition through the air flow system 102. A person skilled in the art will understand that because the illustrated embodiment is schematic in nature, the processing unit 108 can be a separately disposed component of the RSSS 100, or alternatively, it can be disposed within or coupled to and/or on one of the other components of the RSSS 100, such as the gas containing element 106. The processing unit 108 may be more generally part of a controller configured to operate one or more components of the RSSS 100 (e.g., components of the air flow system and/or components of the airway system 104). One exemplary embodiment of a controller that can be used in conjunction with the present disclosure is described below with respect to FIG. 5. In some instances, the processing unit 108 can be interchangeable with a controller, and in other instances the processing unit 108 may be a portion of a controller. Further, one or more processing units 108 and/or one or more controllers can be incorporated into or otherwise used in conjunction with the RSSS 100.

The airway system 104 may include at least one or more conduits 122, 124, 126 that guide the air flow from the air flow system 102 to the ambient environment through at least one exit orifice 128, 130. The conduits 122, 124, 126 may be designed in a manner to mimic, or replicate, the airway anatomy of a human or other animal respiratory system, including the oropharynx, oral, and/or nasal air pathways. The oropharynx, oral, and nasal air pathways may be manufactured by 3D printing based on realistic computed tomography (CT) scan models. Printing material may be selected to mimic the realistic mechanical properties of the airway.

The exit orifice(s) 128, 130 can provide for the release of a mimic exhalation into the ambient environment. The mimic exhalation can include a multiphase turbulent gas cloud. The at least one exit orifice 128, 130 may be configured to represent the shape and/or mechanical properties of a mouth, snout, or beak and surrounding soft tissue where appropriate, and/or it may be configured to represent the shape and/or mechanical properties of a nose or nostrils. In one embodiment, both the shape and/or mechanical properties of a human oropharynx 122 is represented along with the shape and/or mechanical properties of the nose 130 and/or mouth 128 as exit orifices. In the same or a different embodiment, at least one shape of an entire head 138 is represented and contains, entirely or partially, the airway system. Conduits 122, 124, 126 may be fitted in a head 138, which may also be 3D printed. Head 138 may be different face types and geometries such as small, medium, large, long and narrow, or short and wide. The plurality of conduits 122, 124, 126 may also be appropriately scaled versions of the human or other animal respiratory system it is to represent. Such animals may include, but are not limited to, dogs, cats, horses, cows, and birds.

The exit orifice(s), e.g. nose 130 and/or mouth 128, can be configured in such a way to create a range of point-source multiphase gas clouds consistent with a range of natural respiratory emissions. The point-source signifies turbulent flows that take on a shape (e.g., conical) as they advance in distance due, at least in part, to their inherent level of turbulence that results from various factors provided for herein. Thus, as would be understood by a person skilled in the art, in view of the present disclosures, a source (e.g., the nose and/or mouth mimic) that is smaller compared to the scale of motion or evolution of the released flow is provided. The RSSS 100 can be configured such that a range of natural respiratory emissions can be achieved by making adjustments to the exit orifice(s), such as by allowing for adjustments of the exit orifice(s) either manually or automatically, whether by command or in response to certain parameters. Additionally, or alternatively, the RSSS 100 can be configured such that a range of natural respiratory emissions can be achieved by making adjustments to parameters associated with the exhalation event(s), such as the various manipulations of the gas flow through the systems 102 and 104 disclosed herein. These adjustments can cause various resulting gas clouds.

One or more temperature stabilizing units 110 may be positioned along the airway system 104 to maintain the airway system 104 at a body temperature of a human or other animal. At least one of a plurality of conduits 122, 124, 126 may be equipped with at least one sensor 118, the sensor(s) 118 having capabilities as those described above with respect to the air flow system 102 and/or adapted for use in conjunction with the airway system 104. The at least one sensor 118 may send at least one signal to the at least one processing unit 108 to provide information about the status of the airway system 104, and/or to provide a trigger to initiate a gagging event or other exhalation event(s). One example may be that the at least one sensor 118 triggers collapsing one or more of the plurality of conduits 122, 124, 126. The at least one signal sent to the at least one processing unit 108 might also be used to control some property or characteristic of the airway system 104, such as airway system temperature, degree of fluid released or injected into the airway system, and/or composition of liquid released and/or injected into the airway system 104. For example, the processing unit 108 may include or otherwise be in communication with a human exhalation event control system (e.g., a controller, as described in greater detail below with respect to FIG. 5) configured to generate one or more pulses of air from air received from the gas containing chamber 106 and deliver the pulse of air to the airway system 104 at a prescribed air flow profile, and/or air flow pattern commensurate with a desired exhalation event.

Alternatively, a human exhalation event control system may have another processing unit disposed between the air flow system 102 and the airway system 104. Human exhalation event control system may include a pulse generator 140 and/or valve or plurality of valves to control the air pulse at outlet 116. At least one of the plurality of conduits 122, 124, 126 of the airway system 104 can be in communication with at least one liquid reservoir 132. The at least one liquid reservoir 132 can be used to provide a liquid lining to the at least one of the plurality of conduits 122, 124, 126 of the airway system 104 and/or to otherwise provide a liquid phase to the airway system 104. This may be achieved, for example, through gravity driven flows, plug or liquid injection, direct coating and/or other means of providing a liquid phase to the airway system 104.

In some embodiments the at least one liquid reservoir may be realized through at least one cartridge system 134 that may be inserted into a predefined slot of the airway system in communication with the plurality of conduits. Such a cartridge system 134 may contain different liquids of different fluid rheological or other properties. Liquid release from such a cartridge system 134 may be controlled by the processing unit 108, or another control unit (e.g., a controller as described herein, such as with respect to FIG. 5), electronically, optically, mechanically, magnetically, and/or in a number of different ways to produce a liquid of desired rheological properties in the airway system. The cartridge system 134 can include, for example, multiple cartridges that can be called upon based on different sensed parameters, the multiple cartridges providing different fluids, and/or different mixtures of similar fluids, which can be called upon individually and/or in combination as desired.

Alternatively or additionally, liquids from the at least one liquid reservoir 132 might be injected into the airway system from at least one injection port 133. The at least one injection port 133 might be contiguous with at least one of the plurality of conduits in the airway system or might protrude into at least one of the plurality of conduits. In some embodiments the conduits 122, 124, 126 may be lined with a fluid having properties of mucosalivary fluid. In some embodiments of the airway system 104, at least one of the plurality of the conduits 122, 124, 126 may be actuated to rotate around its own axis to achieve a liquid lining of the wall from fluid released. The fluid can be released into the conduits 122, 124, 126 to establish a liquid film from which at least some droplets may be sheared during air flow ejection through the airway system 104. The airway system may further include a fluid outlet, or drainage receptacle, 135 for excess liquid injected into the airway system. Air flow system 102 can be coupled to one of conduits 122, 124, 126 via the outlet 116 to establish fluid communication.

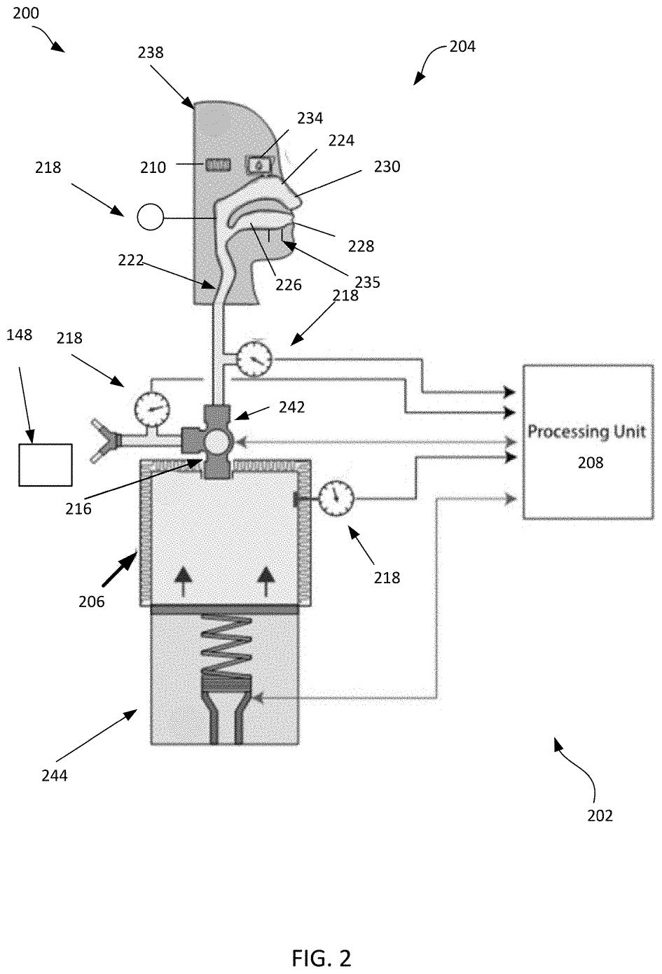

FIG. 2 illustrates another exemplary embodiment of an RSSS 200 including an air flow system 202 and airway system 204. Unless otherwise noted, the operation and components of the airway system 204 are similar to the airway system 104 of FIG. 1. Likewise, reference numbers in the 200 series are representative of the same components in the 100 series in FIG. 1 unless otherwise noted. Accordingly, even if a particular component of the RSSS 200 that is illustrated in FIG. 2 is not introduced in the text below, it is still understood to be a component of the RSSS 200 based on its identification in FIG. 2.

The airway system 204 can include at least one gas containing element 206 and an expulsion element 244 that can provide a particular air volume to the airway system 204. In this embodiment, the gas containing element 206 can be a volumetric element for containing and expelling predefined volumes, although other gas containing element configurations and functionalities are contemplated.

In a method of operation of the volume-based air flow system 202, a prescribed air volume may be loaded into the gas containing element 206 of the air flow system 202. The air volume can then be expulsed from the air flow system 202 and into the airway system 204 in a predetermined manner. As illustrated, the expulsion element may be mechanical, such as a spring-based piston driven expulsion. However, the expulsion may occur through other types of mechanical and/or electro-mechanical actuation, such as an actuation involving a gas, a liquid, and/or a mechanical compression of the at least one gas containing element 206. This actuation may be controlled through appropriate sensors 218 acquiring information during the ejection period, such as flow meters or pressure sensors, which can provide information to at least the processing unit(s) 208 that in turn can modify the action of the mechanical actuation process, including in real-time. The ejection of the air from the gas containing element 206 may occur through an outlet 216 having at least one resistive element whose resistance might be modulated to attain particular air flow characteristics such as velocity profile, peak velocity, ejection duration, peak volumetric flow rate, etc. The at least one resistive element 216 may be purely passive and may or may not exhibit nonlinear pressure-flow characteristics, such as exhibiting flow limitation. Alternatively, the at least one resistive element may be actively controlled (e.g., electrically, magnetically, mechanically, optically, etc.) with linear, nonlinear, and/or time-varying resistive properties. Alternatively, the at least one resistive element may be a combination of at least one active and at least one passive element arranged in parallel or in series.

In some embodiments, outlet 216 may include a resistive element 242 that operates as both an inlet and outlet for the gas containing element 206, such as a three-way valve. The airway system 204 attaches to the air flow system 202 such that the air expelled from the air flow system can be diverted through the airway system 204 and can perform as described above with respect to FIG. 1 to expel the a type of exhalation. In some RSSS systems and methods, an air flow system may include a combination of the pressure-based air flow system of FIG. 1 and the volume-based air flow system in FIG. 2.

In order for RSSS embodiments to mimic, or reproduce, animal, including human, exhalations, the systems can be configured to provide multi-phase turbulent gas cloud emissions in which a warm and moist air is mixed with liquid droplets and solid residues/aerosols from the one or more conduits representing the upper airways. The type of exhalation (e.g., quiet breathing, soft speaking/vocalizations, loud speaking/vocalizations, singing, yelling, coughing or sneezing) desired to be performed by the system determines the momentum of the emitted air flow of the emitted multiphase turbulent cloud and the turbulent regime (as commonly measure in fluid dynamics by the Reynold number) prescribed by the RSSS. Likewise, the type of exhalation determines the range of volumes of air and droplets and/or solid residues/aerosols emitted, the volumetric air flow and velocity of air and droplet and/or solid residue/aerosol emissions, the duration of the emission, the temporal profile of such emissions, and/or the liquid and/or solid volume fractions, among other characteristics of the emission flow. The system can be configured to sense, control, and/or prescribe, these air flows, air flow velocities, air volumes, emission durations, and/or temporal profiles or airflow patterns based, at least in part, on the desired type of exhalation to be performed. In one embodiment, reference values for the profiles of the relevant variables are stored in a lookup table in the processing unit and used to control the air flow and airway systems to achieve the desired emission characteristics. In another embodiment, mathematical equations (deterministic, stochastic, or statistical associations) might be implemented on the processing unit and solved in real time based on sensed variables from the RSSS to determine value of intermediate variables or variables to prescribe and control in the RSSS to achieve a desired exhalation type. For example, the system can mimic a healthy human subject by producing exhalation volume approximately in the range of about 1 L to about 4 L and peak air flow velocity in excess of approximately 100 m/sec, a typical liquid volume fraction of <about 1%. The duration of the exhalation emission can be adjusted approximately in a range between about 100 msec (e.g., sneeze) to about 1 sec to about 2 sec (normal tidal breath). If a more violent type of exhalation, such as sneezes or coughs, are required to be mimicked, the system can employ an asymmetric flow rate profile with a rapid rise in flow rate and slower decay. For example, in mimicking a cough, the peak flow rates—depending on size, age, gender—may be approximately in the range of about 1 L/s to about 20 L/s with the peak flow rates reached rapidly over rise times ranging from dozens to hundreds of milliseconds and a decay time of approximately 1.5 seconds. This sharp rise allows a user to capture multi-phase cloud dynamics and create droplets from the fluid lining, as well as entrap them in the cloud as the multi-phase cloud exits the airway system. These characteristics may be further refined based on species, body size, age, shape of organism, and/or health, medical conditions, and/or disease states of the organism, among other factors.

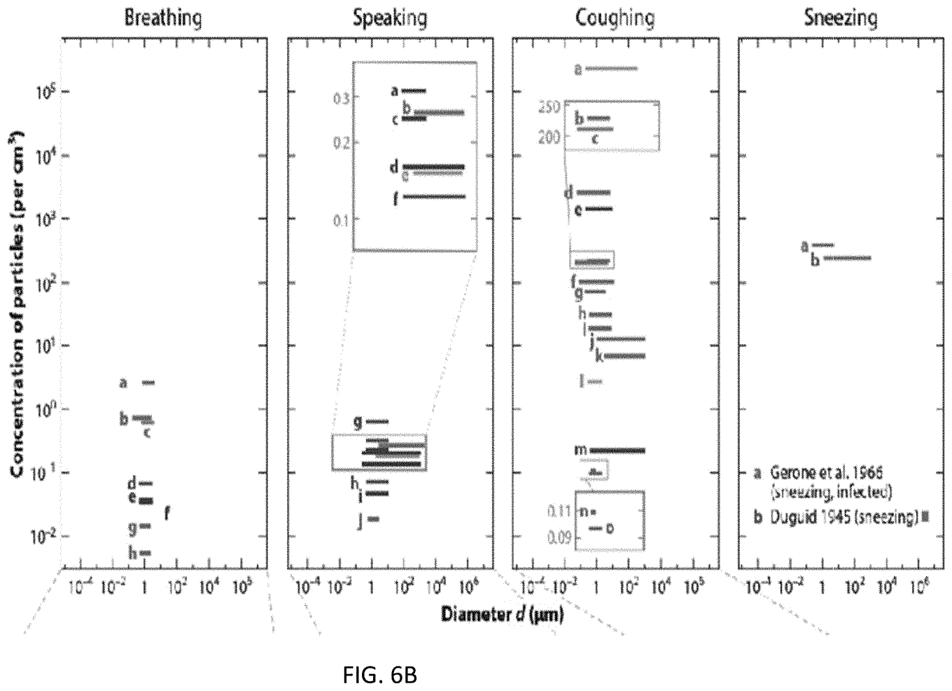

The liquid phase of the multiphase turbulent gas cloud emission can be largely determined by the interaction of the exhalation air flow with the liquid lining of the respiratory system as the shear forces acting on the liquid lining destabilize the liquid layer, leading to droplet formation and emission alongside the warm and humid exhalation air. The liquid of some of the droplets so formed can evaporate quickly, leaving behind a solid droplet residue or aerosol that may also be emitted during the exhalation or may form at various stages after the emission of the multiphase turbulent exhalation cloud into the ambient environment. Depending on the exhalation type, droplets can range in diameter from <1 μm to about 5 mm with more forceful or violent exhalations typically exhibiting a larger range in the size distributions as seen, for example, in FIGS. 6A-B. As illustrated, FIG. 6A is a graph of droplet size distributions from the comparing respiratory emissions under a range of conditions; measured with different instrumentation and at different distances from the mouth from breathing (nose and mouth), speaking, coughing, and sneezing; and examining both infected and healthy subjects. FIG. 6B shows graphs illustrating the relationship of concentration linked to choices of volume fraction of liquid in gas phase for the multiphase emission for breathing, speaking, coughing and sneezing emissions.

The number and speed of droplets the system generates can be based on at least one or more of the air flow pattern, the magnitude of the air flow, the geometry of the airway, the mechanical properties of the mouth and/or nose, and on rheological properties of the liquid lining the airways. The rheological properties of biological fluids can be determined in large part by their biomolecule and solute composition and concentrations, which can determine characteristics such as shear viscosity, extensional viscosity, and/or surface tension. These concentrations can vary across species, developmental stage of an organism, diet, and/or in health and/or disease. Human mucosalivary fluid, for example, typically has a shear viscosity approximately in the range of about 10 mPa·s to about 1000 mPa·s, an extensional viscosity of up to about 1000 ms, and a surface tension approximately in the range of about 50 mN/m to about 80 mN/m for example. Therefore, fluids used in the disclosed RSSS may be selected that have properties within the range of human mucosalivary fluid. The rheological properties (e.g., elastic, viscosity, surface tension) of the liquid of a range of rheological properties mimic bodily liquids in health or disease. The fluids may be seeded with tagging labels, such as tagged DNA, tagged particles or dye to determine the spatial distribution of mimic exhalation emissions, for example. In some embodiments the liquid may be seeded with microorganisms. The droplets and/or solid residues/aerosols formed in the multi-phase air flow and emitted in a multi-phase cloud emission, or formed upon emission can be characterized, for example, by their respective size and/or speed distributions upon exit and/or at various distances from the airway opening/point of emission.

For given fluid rheological properties, level of coating of the airway system, and type of exhalation, the air flow velocity, total air volume, and duration, rise and decay times of the air flow system will be prescribed (through a lookup table, or mechanistic/deterministic/statistical mathematical relationships implemented on the processing unit) to achieve a desired fluid fragmentation regime and liquid volume fraction at the airway opening. In one embodiment, each of the plurality of valves/resistive elements 116 can be programmed in such a manner to excite different modes of fluid fragmentation and hence be used to tune the droplet size distribution to desired levels. The opening/closing/duty cycle and the timing of each valve in a parallel arrangement of valves that feed the airway system can be controlled so as to drive the fluid layer in the airway system into different instabilities that in turn provide characteristic droplet sizes of emission events. In another embodiment, the fluid is injected into the airway system and through a combination of fluid fragmentation and droplet entrainment, a droplet size distribution is generated by prescribing the air flow velocity, total air volume, and duration, rise and decay times of the air flow along with the duration and fluid volume injected into the airway.

The evolution of the multiphase exhalation cloud in the ambient environment depends, at least in part, on the type of emission and the ambient environmental conditions (e.g., temperature, humidity, and ambient air flow conditions), as well as the temperature and humidity of the multiphase exhalation cloud, which may be controlled to be injected into the ambient environment fully or close-to fully humidified (e.g., approximately in the range of about 75% to about 100% relative humidity) and at body temperature. For humans, core body temperature typically ranges from about 36.5° C. to about 37.5° C., though can fall significantly below this range in hypothermic conditions or exceed this range in hyperthermic or hyperpyrexic conditions. Therefore, disclosed airway systems may be maintained at human or other animal body temperatures. Other normative reference ranges exist for other endothermic organisms and exothermic organisms. Together with the conditions of the ambient environment, the relative humidity, temperature, and/or liquid volume fraction of the multiphase turbulent exhalation cloud determine the cloud's buoyancy characteristics in the environment and therefore contribute to the range and distance of the exhalation cloud into the environment. Therefore, disclosed RSSS systems may be set to achieve a desired buoyancy by controlling the relative density difference between the emitted multiphase cloud and the environment, or ambient air.

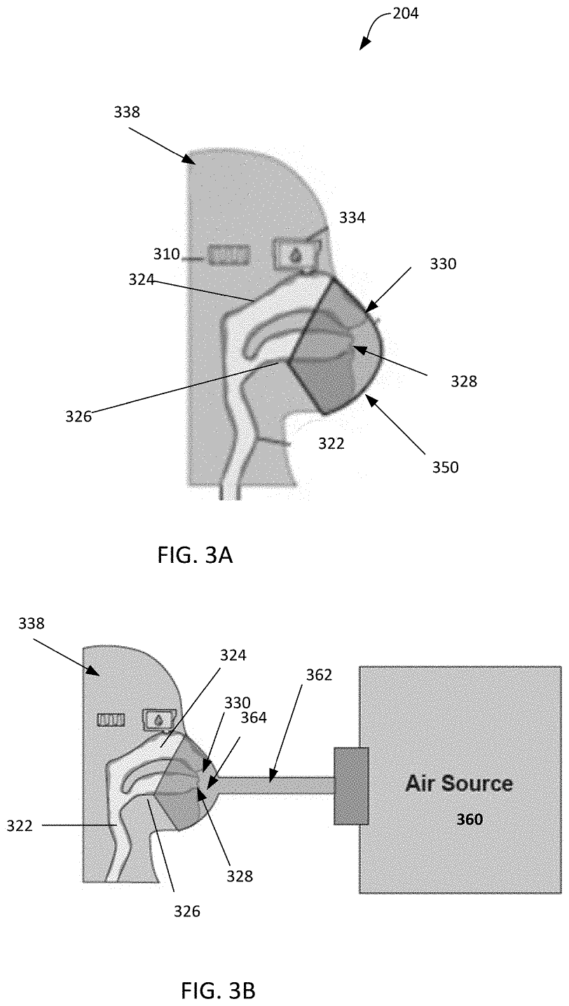

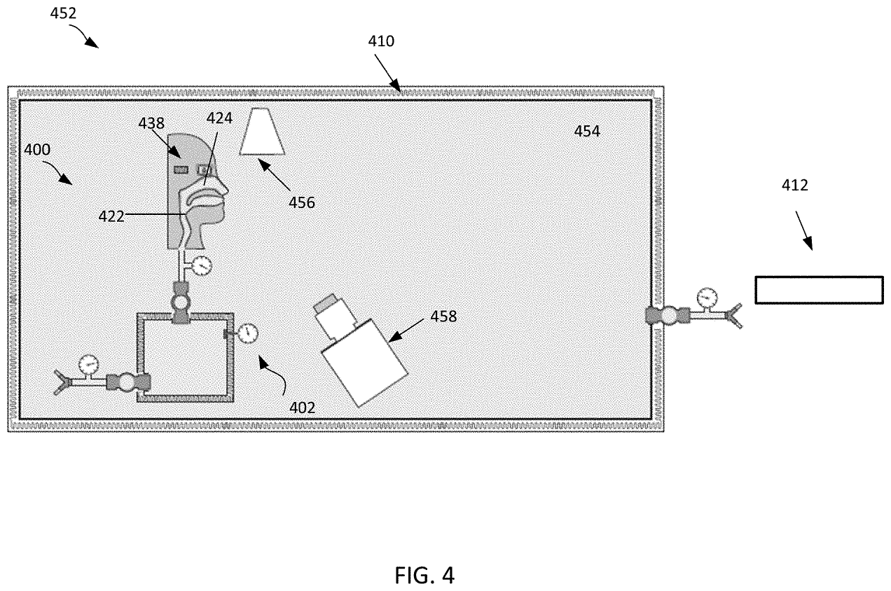

FIGS. 3A-3B illustrate exemplary embodiments of an airway system 304 in testing configuration. Unless otherwise noted, the operation and components of the airway system 304 are similar to the airway system 104 of FIG. 1 and/or the airway system 204 of FIG. 2. Likewise, reference numbers in the 300 series are representative of the same components in the 100 series in FIG. 1 and/or the 200 series in FIG. 2 unless otherwise noted. Accordingly, even if a particular component of the RSSS 300 that is illustrated in FIGS. 3A-3B is not introduced in the text below, it is still understood to be a component of the RSSS 300 based on its identification in FIGS. 3A-3B.

As illustrated in FIG. 3A, the shape of an entire head 338 may be used to fit protective equipment 350, such as masks or face shields, to the head 338 covering outlets 328, 330. FIG. 3B shows an entire head 338 equipped with medical devices such as a continuous airway pressure mask 364 coupled to a high pressure air source 360 via a cannula 362. Heads 338 may also be equipped with other medical devices, such as a high-flow nasal cannula. In addition, the RSSS 300 integrated with the cannula 362 can also be used to test the effectiveness of pressurized air and/or drug delivery into a respiratory system of a human or other animal. In some embodiments, the bottom of the airway system, in this case conduit 322, may be connected with an additional flow sensor, connected to a mass spectrometer (MS) and/or gas chromatography (GC) system, and/or particle sensor (connected to a particle spectrometer, for example) for characterization of flow volume and/or concentration of any specific particle of interests imposed by the cannula. RSSS 300 with the entire head 338 may further be used to perform mock intubations or dental procedures. The airway system 304 of FIGS. 3A-3B may be incorporated into any of the disclosed RSSSs, or any such RSSS derivable from the present disclosures. The RSSS may be fitted with fabric holder configured to hold at least one of a protective device or a fabric for use as part of a protective device. Such embodiments may be used to study the degree to which such protective or medical devices or procedures may reduce or amplify the emission of respiratory droplets into the ambient environment, as well as back-flow induced aerosolization due to pressurized air delivery into the nasal and oral cavities, and lack of seal of face mask or cannula.

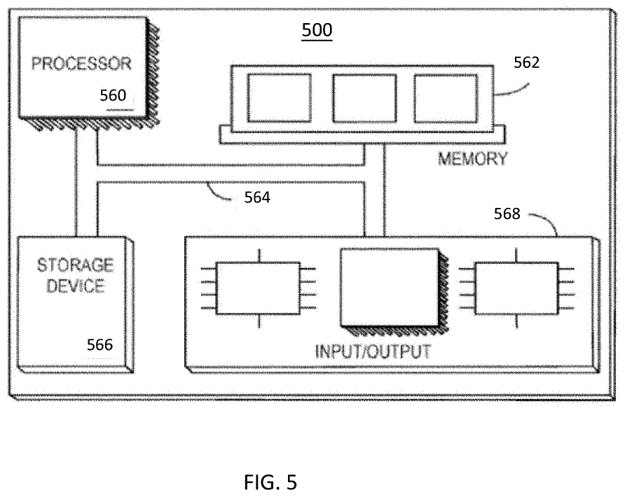

FIG. 4 illustrates an RSSS 400 in a test environment 452. Unless otherwise noted, the operation and components of the RSSS 400 are similar to the RSSS 100 of FIG. 1, the RSSS 200 of FIG. 2, and the RSSS 300 of FIGS. 3A-3B. Accordingly, reference numbers in the 400 series are representative of the same components in the 100 series in FIG. 1, the 200 series in FIG. 2, and/or the 300 series in FIGS. 3A-3B unless otherwise noted. Accordingly, even if a particular component of the RSSS 400 that is illustrated in FIG. 4 is not introduced in the text below, it is still understood to be a component of the RSSS 400 based on its identification in FIG. 4.

The test environment 452 may include a full environmental control space 454 and an optical system 456 for characterization of protective equipment effectiveness, fit and source protection for multiple head geometries, and/or quantification of various respiratory emission levels, under various environmental temperatures and room humidities. The RSSS 400 can include an airflow system 402 and an airway system 404. As illustrated, the airway system 404 can include a full head 438 can be fitted on the airway formed by conduits 422, 424, 426 and outlets 428, 430. The air flow system 402, while illustrated as a pressure-based type, may be any of the air flow systems or combinations described herein or otherwise known and adaptable to the present disclosures by those skilled in the art, in view of the present disclosures. The integrated RSSS 400 may be positioned within a temperature/humidity control chamber or other environment controlled space 454. A temperature stabilizing unit 410 and humidifier/dehumidifier 412 can regulate temperature and/or humidity, respectively, in the environmental control space 454, each having respective temperature and humidity controllers. Lights 456, such as super-bright LED light sources or monochromatic-laser sources/sheets, may be placed around the RSSS 400, illuminating the area around the mouth outlet 428. An analysis apparatus, such as a camera 458 or slow-motion video camera, or other particle sensors may be positioned at a suitable location, as shown on the side of the RSSS 400, to visualize and/or record the respiratory emission controlled. In some embodiments, the recorded video may be simultaneously uploaded to a computer with advanced image processing, which can allow the precise evaluation and/or quantification of the level of emission control of different source protection devices, worn on various head and face geometries at different environmental conditions, such as the processing system disclosed in FIG. 5. The airflow system 404 may be fitted with protective equipment or medical devices as described in FIGS. 3A-3B to test the efficacy of these equipment and devices. Additionally, a fabric or other material device may be placed close to an exit orifice, for example to collect droplets or aerosol from the emission for further virus tests to examine the concentration of pathogens inside, and/or to test the tendency of the material to absorb respiratory droplets and/or to inactivate microorganisms seeded into the multiphase exhalation cloud. Multiple RSSSs may be placed in a single test environment, such as two or more heads facing each other, with or without protective equipment, to characterize the effectiveness of protective equipment and measure any leakage around the protective equipment that may occur during an exhalation event.

Disclosed RSSS embodiments may also be used to test the propensity of medical equipment or interventions such as continuous positive airway pressure, dental procedures, high-flow nasal cannula, intubation, etc., to promote generation of respiratory droplets and/or aerosols/solid residues that might be vehicles for pathogenic payload. Additionally, an RSSS can be used to test the efficacy of ventilation, air filtration, and/or flow diversion strategies to mitigate the effects of exhalations and the attendant droplet and/or solid residue/aerosol transport, depositions, or suspensions in the ambient air of hospital rooms, office buildings, retirement homes, assisted living facilities, factories, shops, auditoriums, classrooms, lecture halls or other residential, commercial, or professional dwellings/environments, or in transportation systems, such as gondolas, cars, buses, subway carriages, trains, boats/ships, or aircraft. Likewise, the efficacy of such ventilation, air filtration systems, and/or flow diversion strategies to mitigate transmission may also be of interest in a wide range of indoor and outdoor animal husbandries and/or battery cage conditions in the livestock industry or veterinary facilities, including medical research facilities where animal experiments might be conducted.

Disclosed RSSS embodiments can be operated in a vertical manner in which the emission takes place horizontally, while in another embodiment, the RSSS can be operated in a horizontal manner in which the emission takes place vertically. In still another embodiment, the orientation of the RSSS, and hence the direction of emission, may be adjustable continuously between the vertical and horizontal. Disclosed embodiments of RSSSs may be configured to fit into a biosafety cabinet or other confined chamber. RSSSs may be portable and/or battery operated.

FIG. 5 is a block diagram of one exemplary embodiment of a computer system 500 upon which the present disclosures can be built, performed, trained, tested, optimized, etc. For example, the implementation of designing and/or modeling RSSSs, in view of the above described parameters to accurately and consistently produce exhalation emissions, as well as other features disclosed herein, can be performed by a system 500. The system 500 can include a processor 560, a memory 562, a storage device 566, and an input/output device 568. Each of the components 560, 562, 566, 568 can be interconnected, for example, using a system bus 564. The processor 560 can be capable of processing instructions for execution within the system 500, such as iterating various exhalation events based on inputs related to the one or more parameters of exhalation patterns/profiles. The processor 560 can be a single-threaded processor, a multi-threaded processor, or similar device. The processor 560 can be capable of processing instructions stored in the memory 562 or on the storage device 566. The processor 560 may execute operations such as adjusting the parameters such as temperature, humidity, velocity, durations, and/or temporal profiles based, at least in part, on the desired type of exhalation to be performed including fixing one or more of such variables while manipulating other such variables based on input provided by the system and/or a user(s). A person skilled in the art, in view of the present disclosures, will understand various ways by which the processor 560 can be programmed to design, model, test, optimize, and/or manufacture RSSSs.

The memory 562 can store information within the system 500. In some implementations, the memory 562 can be a computer-readable medium. The memory 562 can, for example, be a volatile memory unit or a non-volatile memory unit. In some implementations, the memory 562 can store information related to various emitter designs and variables associated with types of exhalation emissions.

The storage device 566 can be capable of providing mass storage for the system 500. In some implementations, the storage device 566 can be a non-transitory computer-readable medium. The storage device 566 can include, for example, a hard disk device, an optical disk device, a solid-date drive, a flash drive, magnetic tape, and/or some other large capacity storage device. The storage device 566 may alternatively be a cloud storage device, e.g., a logical storage device including multiple physical storage devices distributed on a network and accessed using a network. In some implementations, the information stored on the memory 562 can also or instead be stored on the storage device 566.

The input/output device 568 can provide input/output operations for the system 500. In some implementations, the input/output device 568 can include one or more of network interface devices (e.g., an Ethernet card), a serial communication device (e.g., an RS-232 10 port), and/or a wireless interface device (e.g., a short-range wireless communication device, an 802.7 card, a 3G wireless modem, a 4G wireless modem, a 5G wireless modem). In some implementations, the input/output device 540 can include driver devices configured to receive input data and send output data to other input/output devices, e.g., a keyboard, a printer, and/or display devices. In some implementations, mobile computing devices, mobile communication devices, and other devices can be used.

In some implementations, the system 500 can be a microcontroller. A microcontroller is a device that contains multiple elements of a computer system in a single electronics package. For example, the single electronics package could contain the processor 560, the memory 562, the storage device 566, and/or input/output devices 568.

Although an example processing system has been described above, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine-readable storage device, a machine-readable storage substrate, a memory device, a composition of matter effecting a machine-readable propagated signal, or a combination of one or more of them.

Various embodiments of the present disclosure may be implemented at least in part in any conventional computer programming language. For example, some embodiments may be implemented in a procedural programming language (e.g., “C”), or in an object-oriented programming language (e.g., “C++”). Other embodiments may be implemented as a pre-configured, stand-along hardware element and/or as preprogrammed hardware elements (e.g., application specific integrated circuits, FPGAs, and digital signal processors), or other related components.

The term “computer system” may encompass all apparatus, devices, and machines for processing data, including, by way of non-limiting examples, a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Such implementation may include a series of computer instructions fixed either on a tangible, non-transitory medium, such as a computer readable medium. The series of computer instructions can embody all or part of the functionality previously described herein with respect to the system. Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM and DVD-ROM disks. The processor and the memory can be supplemented by, or incorporated in, special purpose logic circuitry. The components of the system can be interconnected by any form or medium of digital data communication, e.g., a communication network. Examples of communication networks include a local area network (“LAN”) and a wide area network (“WAN”), e.g., the Internet.

Those skilled in the art should appreciate that such computer instructions can be written in a number of programming languages for use with many computer architectures or operating systems. Furthermore, such instructions may be stored in any memory device, such as semiconductor, magnetic, optical, or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the present disclosure may be implemented as a combination of both software (e.g., a computer program product) and hardware. Still other embodiments of the present disclosure are implemented as entirely hardware, or entirely software.

Examples of the above described systems and methods can include the following.

1. A system for exhalation emission, comprising:

an air flow system configured to have a gas flow exit therefrom, the gas flow having a prescribed flow pattern and a prescribed momentum corresponding to one or more exhalation events; and

an airway system having an airway in fluid communication with the air flow system to receive the gas flow from the air flow system and at least one exit orifice, the airway system configured to mimic a respiratory tract,

wherein the system for exhalation emission is configured such that at least some portion of the gas flow produced from the air flow system passes through the airway and out of the at least one exit orifice in a multiphase gas cloud, the multiphase gas cloud comprising at least some portion of the gas flow produced from the air flow system and one or more of a plurality of droplets, solid residues, or aerosols.

2. The system of claim 1, wherein the one or more of the plurality of droplets, solid residues, or aerosols are produced by the airway system due to at least one of the gas flow interacting with a liquid coating disposed in the airway in conjunction with mimicking a respiratory tract or the gas flow interacting with liquid injected into the airway system.
3. The system of claim 1, wherein the air flow system further comprises a gas containing chamber having at least one of a temperature stabilizing unit coupled to an exterior of the gas containing chamber and configured to place the gas flow in a prescribed temperature range and a humidifier/dehumidifier system configured to place the gas flow in a prescribed humidity range.
4. The system of claim 3, wherein the air flow system further comprises at least one pressure or flow regulator configured to deliver input air into the gas containing chamber.
5. The system of any of claims 1 to 3, wherein the air flow system further comprises:

an exhalation event control system disposed between at least one of the air flow system and the airway system, the exhalation event control system being configured to generate a pulse of air from air received from a gas containing chamber and deliver the pulse of air to the airway system at the prescribed flow pattern.

6. The system of claim 5, wherein the prescribed flow pattern comprises at least one of a velocity of air flow, a duration of air flow, or a magnitude modulation of air flow commensurate with a desired exhalation event from the one or more exhalation events.
7. The system of claim 5 or 6, wherein the exhalation event control system is further configured to deliver the pulse of air to the airway system at a prescribed volume.
8. The system of any of claims 5 to 7, wherein the exhalation event control system comprises at least one of a pulse generator or a valve.
9. The system of any of claims 1 to 7, wherein the airway system further comprises a fluid disposed in the airway, the fluid having rheological properties akin to a mucosalivary fluid.
10. The system of claim 9, wherein the fluid lines at least a portion of the airway, the fluid being configured to be sheared by the gas flow produced from the air flow system that passes through the airway such that the fluid becomes the one or more of the plurality of droplets, the solid residues, or the aerosols in the multiphase gas cloud.
11. The system of claim 9 or 10, further comprising:

a fluid inlet in fluid communication with the airway; and

a fluid outlet in fluid communication with the airway,

wherein the system is configured to pass the fluid configured to mimic mucosalivary fluid from the inlet, into the airway, and out of the outlet.

12. The system of any of claims 1 to 10, wherein the at least one exit orifice comprises at least two exit orifices.
13. The system of claim 12, wherein a first exit orifice of the at least two exit orifices is configured to mimic at least one of a shape or mechanical properties of a human nose and a second exit orifice of the at least two exit orifices is configured to mimic at least one of a shape or mechanical properties of a human mouth.
14. The system of any of claims 1 to 13, wherein the at least one exit orifice comprises a mouthpiece configured to mimic at least one of a shape or mechanical properties of a human mouth.
15. The system of any of claims 1 to 14, wherein the one or more human exhalation events comprises at least one of coughing, sneezing, breathing, talking, panting, gagging, or singing.
16. The system of claim 15, wherein the one or more human exhalation events comprises at least one of coughing or sneezing.
17. The system of any of claims 1 to 16, wherein the one or more exhalation events are further configured based on at least one of different ages, different sizes, different shapes, or different medical conditions.
18. The system of any of claims 1 to 17, wherein the air flow system is configured to achieve a prescribed buoyancy of the multiphase gas cloud a certain distance from the airway system.
19. The system of any of claims 1 to 18, wherein the least one exit orifice is configured to replicate at least one of a shape or mechanical properties of at least one of a nose or a mouth.
20. The system of any of claims 1 to 19, wherein liquids of a range of rheological properties are configured to be loaded into the airway system through at least one liquid inlet.
21. The system of claim 20, wherein rheological properties of the liquid are in a range of rheological properties of bodily liquids in health or disease.
22. The system of any of claims 1 to 21, in which at least one of the airway and the at least one exit orifice is configured in such a way to create a range of point-source multiphase gas clouds consistent with a range of natural respiratory emissions.
23. The system of claim 1, wherein the air flow system is configured to achieve a range of size distributions for at least one of the plurality of droplets, the solid residue, or the aerosol upon exit of the airway system.
24. A method for generating an exhalation emission, comprising:

generating a flow of air at a prescribed flow pattern, a prescribed momentum, and one or both of a prescribed liquid or solid volume fraction, for one or more exhalation events; and

directing the flow of air through an airway configured to mimic a respiratory tract such that at least some air of the flow of air exits the airway in a multiphase gas cloud, the multiphase gas cloud comprising the at least some air and one or more of a plurality of droplets, solid residues, or aerosols.

25. The method of claim 24, wherein directing the flow of air through an airway configured to mimic a respiratory tract further comprises causing at least one of a fluid disposed in the airway to become entrained with the at least some air of the flow of air such that the fluid becomes the one or more of the plurality of droplets, solid residues, or aerosols, or injecting a fluid into the respiratory tract such that the injected fluid interacts with the flow of air to become the one or more of the plurality of droplets, solid residues, or aerosols.
26. The method of claim 24,

wherein the fluid disposed in the airway lines at least a portion of the airway, and

wherein directing the flow of air through an airway configured to mimic a respiratory tract further comprises shearing the fluid away from the airway to cause the fluid to become entrained with the at least some air of the flow of air.

27. The method of claim 25 or 26, further comprising passing the fluid through the airway.
28. The method of any of claims 24 to 27, further comprising adjusting at least one of a pressure of the flow of air, the temperature of the flow of air, or the humidity of the flow of air to fall within a respective prescribed pressure range, a prescribed temperature range, or a prescribed humidity ranged based on data related to at least one of an age of the human or animal, a size of the human or animal, a shape of a human or animal, or one or more medical conditions of a human or animal.
29. The method of any of claims 24 to 28, further comprising adjusting at least one of peak flow velocity of the flow of air, a duration of the flow of air, or a volume of the flow of air based on data related to at least one of an age of the human or animal, a size of the human or animal, a shape of a human or animal, or one or more medical conditions of a human or animal.
30. The method of any of claims 24 to 29, wherein the at least some air of the flow of air that exits the airway in a gas cloud exits through at least two exit orifices.
31. The method of claim 30, wherein the at least two exit orifices comprises a first exit orifice configured to replicate at least one of a shape or mechanical properties of a nose and a second exit orifice configured to replicate at least one of a shape or mechanical properties of a mouth.
32. The method of any of claims 24 to 31, wherein the prescribed pressure range, the prescribed temperature range, and the prescribed humidity range of the generated flow of air are prescribed based on one or more intended exhalation events.
33. The method of claim 32, wherein the one or more intended exhalation events comprises at least one of coughing, sneezing, breathing, talking, gagging, panting, or singing.

One skilled in the art will appreciate further features and advantages of the disclosure based on the above-described embodiments. Accordingly, the disclosure is not to be limited by what has been particularly shown and described, except as indicated by the appended claims. All publications and references cited herein are expressly incorporated herein by reference in their entirety.

1. A system for exhalation emission, comprising:
an air flow system configured to have a gas flow exit therefrom, the gas flow having a prescribed flow pattern and a prescribed momentum corresponding to one or more exhalation events; and
an airway system having an airway in fluid communication with the air flow system to receive the gas flow front the air flow system and at least one exit orifice, the airway system configured to mimic a respiratory tract,
wherein the system for exhalation emission is configured such that at least some portion of the gas flow produced from the air flow system passes through the airway and out of the at least one exit orifice in a multiphase gas cloud, the multiphase gas cloud comprising at least some portion of the gas flow produced from the air flow system and one or more of a plurality of droplets, solid residues, or aerosols.

2. The system of claim 1, wherein the one or more of the plurality of droplets, solid residues, or aerosols are produced by the airway system due to at least one of the gas flow interacting with a liquid coating disposed in the airway in conjunction with mimicking a respiratory tract or the gas flow interacting with liquid injected into the airway system.

3. The system of claim 1, wherein the air flow system further comprises a gas containing chamber having at least one of a temperature stabilizing unit coupled to an exterior of the gas containing chamber and configured to place the gas flow in a prescribed temperature range and a humidifier/dehumidifier system configured to place the gas flow in a prescribed humidity range.

4. The system of claim 3, wherein the air flow system further comprises at least one pressure or flow regulator configured to deliver input air into the gas containing chamber.

5. The system of claim 1, wherein the air flow system further comprises:
an exhalation event control system disposed between at least one of the air flow system and the airway system, the exhalation event control system being configured to generate a pulse of air from air received from a gas containing chamber and deliver the puke of air to the airway system at the prescribed flow pattern.

6. The system of claim 5, wherein the prescribed flow pattern comprises at least one of a velocity of air flow, a duration of air flow, or a magnitude modulation of air flow commensurate with a desired exhalation event from the one or more exhalation events.

7. The system of claim 5, wherein the exhalation event control system is further configured to deliver the pulse of air to the airway system at a prescribed volume.

8. The system of claim 5, wherein the exhalation event control system comprises at least one of a pulse generator or a valve.

9. (canceled)

10. (canceled)

11. (canceled)

12. (canceled)

13. (canceled)

14. (canceled)

15. The system of claim 1, wherein the one or more human exhalation events comprises at least one of coughing, sneezing, breathing, talking, panting, gagging, or singing.

16. (canceled)

17. The system of claim 1, wherein the one or more exhalation events are further configured based on at least one of different ages, different sizes, different shapes, or different medical conditions.

18. The system of claim 1, wherein the air flow system is configured to achieve a prescribed buoyancy of the multiphase gas cloud a certain distance from the airway system.

19. The system of claim 1, wherein the least one exit orifice is configured to replicate at least one of a shape or mechanical properties of at least one of a nose or a mouth.

20. The system of claim 1, wherein liquids of a range of rheological properties are configured to be loaded into the airway system through at least one liquid inlet.

21. The system of claim 20, wherein rheological properties of the liquid are in a range of rheological properties of bodily liquids in health or disease.

22. (canceled)

23. (canceled)

24. A method for generating an exhalation emission, comprising:
generating a flow of air at a prescribed flow pattern, a prescribed momentum, and one or both of a prescribed liquid or solid volume fraction, for one or more exhalation events; and
directing the flow of air through an airway configured to mimic a respiratory tract such that at least some air of the flow of air exits the airway in a multiphase gas cloud, the multiphase gas cloud comprising the at least some air and one or more of a plurality of droplets, solid residues, or aerosols.

25. The method of claim 24, wherein directing the flow of air through an airway configured to mimic a respiratory tract further comprises causing at least one of a fluid disposed in the airway to become entrained with the at least some air of the flow of air such that the fluid becomes the one or more of the plurality of droplets, solid residues, or aerosols, or injecting a fluid into the respiratory tract such that the injected fluid interacts with the flow of air to become the one or more of the plurality of droplets, solid residues, or aerosols.

26. The method of claim 25,
wherein the fluid disposed in the airway lines at least a portion of the airway, and
wherein directing the flow of air through an airway configured to mimic a respiratory tract further comprises shearing the fluid away from the airway to cause the fluid to become entrained with the at least some air of the flow of air.

27. (canceled)

28. The method of claim 24, further comprising adjusting at least one of a pressure of the flow of air, the temperature of the flow of air, or the humidity of the flow of air to fall within a respective prescribed pressure range, a prescribed temperature range, or a prescribed humidity ranged based on data related to at least one of an age of the human or animal, a size of the human or animal, a shape of a human or animal, or one or more medical conditions of a human or animal.

29. The method of claim 24, further comprising adjusting at least one of peak flow velocity of the flow of air, a duration of the flow of air, or a volume of the flow of air based on data related to at least one of an age of the human or animal, a size of the human or animal, a shape of a human or animal, or one or more medical conditions of a human or animal.

30. The method of claim 24, wherein the at least some air of the flow of air that exits the airway in a gas cloud exits through at least two exit orifices.

31. (canceled)

32. (canceled)

33. (canceled)
